(19) United States
(12) Patent Application Publication
(10) Pub. No.: US 2012/0258171 A1
(43) Pub. Date: Oct. 11, 2012

(54) COMPOSITION COMPRISING FESOTERODINE AND FIBER

(75) Inventors: Daniela Stumm, Berlin (DE); Katrin Rimkus, Iserlohn (DE)

(73) Assignee: ratiopharm GmbH, Ulm (DE)

(21) Appl. No.: 13/501,489

(22) PCT Filed: Oct. 28, 2010
(86) PCT No.: PCT/EP10/06594

§ 371 Date: Jun. 21, 2012

(30) Foreign Application Priority Data
Oct. 30, 2009 (EP) 09013696.1

(51) Int. Cl. A61K 31/222; A61K 9/28; A61P 13/10

(52) U.S. Cl. 424/465; 514/546; 427/2.14

(57) ABSTRACT

The invention relates to a pharmaceutical composition containing (a) fesoterodine and/or fesoterodine metabolites and (b) fibers, wherein the weight ratio of components (a):(b) is in the range from 1:50 to 1:2; and oral dosage forms containing the pharmaceutical composition. The invention further relates to dry methods of preparing those dosage forms.

The invention relates to a pharmaceutical composition containing in particular (a) fesoterodine and/or fesoterodine metabolites and (b) fibers, and also oral dosage forms containing the pharmaceutical composition. The invention further relates to dry methods of preparing those dosage forms. Finally, the invention relates to the use of vegetable fibers for preparing a pharmaceutical formulation with modified release for the treatment of an overactive bladder.

Fesoterodine is an antimuscarinic agent for the treatment of an overactive bladder. When treated with fesoterodine, the symptoms of an overactive bladder, which patients found very troublesome, were improved considerably. In all the clinically relevant end points in both Phase III studies (2, 3) (urge incontinence events/24 h, frequency of micturition, mean micturition volume), statistically significant improvements over a placebo were achieved. Fesoterodine is currently marketed under the trade name Toviaz®.

The IUPAC name of fesoterodine [INN] is 2-[(1R)-3-(di-isopropylamine)-1-phenylpropyl]-4-(hydroxymethyl)phenyl-isobutyrate. The chemical structure of fesoterodine is shown in formula (1) below:

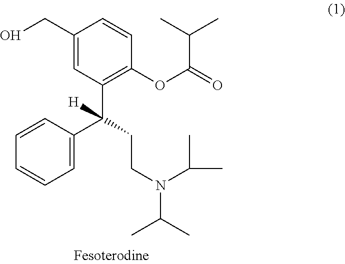

Synthesis pathways for fesoterodine can be derived from EP 1 077 912 B1. Salts of fesoterodine are described in EP 1 230 209 B1.

Fesoterodine is not particularly stable against hydrolysis. Taking this fact into account, WO 2007/141298 proposed fesoterodine tablet formulations containing an active agent and a stabiliser against hydrolysis, the stabiliser preferably being xylitol. In addition, the active agent had to be incorporated into a matrix or artificial polymer so that extended release could be achieved. It was also found that the amount of decomposition products was only advantageous if the formulations proposed were prepared by means of classic wet granulation. In an identical composition, direct compression or even dry granulation led to considerably larger amounts of undesirable decomposition products (compared to wet granulation).

The production methods described in the state of the art therefore prefer a classic wet granulation process. That is economically complex and expensive and should be avoided. Furthermore, in the course of wet granulation, the active agent usually comes into contact with solvents for a lengthy time. This, too, should be avoided.

The formulations proposed in the state of the art also require various types of additives (xylitol on the one hand, and retarding polymers on the other) for moisture protection and retardation. Several processing steps are also required for production. It was an object of the present invention, on the other hand, to provide a formulation in which protection against hydrolysis and retardation can be achieved with only one type of additive if possible and with only one processing step if at all possible.

In order to achieve the desired delayed release, the formulations proposed in the state of the art require a large amount of polymer. As a result, only a relatively small content of active agent (drug load) is possible. The formulations described in WO 2007/141298, for example, have a fesoterodine content of 5% by weight or less. A further object of the invention was therefore to provide fesoterodine in a form which also makes a formulation with a high content of active agent possible, preferably with a content of active agent of more than 5%.

A further problem with regard to the tablets described in the state of the art is the fact that a considerable part of the active agent (approx. 20%) is not released as a rule. It was therefore an object of the present invention to provide a dosage form with modified release, wherein the active agent should be released as completely as possible.

Antimuscarinic agents such as fesoterodine are used in treating an overactive bladder. This indication requires patients to have the dosage forms with them at all times. The Toviaz® tablets currently on the market, however, only possess storage stability up to 25° C. This is unsatisfactory particularly in the summer months. A further object of the invention was therefore to provide active agents for the treatment of an overactive bladder, preferably antimuscarinic agents such as fesoterodine, in a form which is suitable for a formulation with a storage stability in practical use of up to 30° C.

In addition, it was an object of the invention to provide a pharmaceutical dosage form for the treatment of an overactive bladder which possesses substantially the same solubility as the formulations described in WO 2007/141298, especially the example formulations shown in Table 1, and is subsequently substantially bioequivalent to them in the case of oral administration.

Finally, it must be noted that from the toxicological point of view, fesoterodine is a very active drug, since it is rapidly and more or less completely activated in the body by unspecific esterases. Hence, it was an object of the invention to provide a “safe” fesoterodine formulation, in which too rapid a rise in concentration is prevented.

It was unexpectedly possible to achieve the above-mentioned objectives by means of a combination of fesoterodine with fibers, and by the use of fibers in formulating active agents for the treatment of an overactive bladder.

The subject matter of the invention is therefore a pharmaceutical composition containing
(a) fesoterodine and/or fesoterodine metabolites, and
(b) fibers,
   wherein the weight ratio of components (a):(b) is preferably in the range from 1:50 to 1:2.

The subject matter of the invention is also a process for producing oral dosage forms, especially tablets, comprising the steps of:

(i) mixing
a) fesoterodine and/or fesoterodine metabolites,
b) fibers with pharmaceutical excipients,
   and optionally further pharmaceutical excipients;
   (ii) compressing the mixture into tablets, optionally with the addition of further pharmaceutical excipients; and
   (iii) optionally film-coating the tablets.

In addition, tablets obtainable by the method of the invention are a subject matter of the invention.

Finally, one subject matter of the invention is the use of fibers for preparing a pharmaceutical formulation with modified release for the treatment of an overactive bladder.

Fesoterodine is a prodrug. After oral ingestion, esterases cause the prodrug to be activated in the human body into the active metabolite. The present invention relates to fesoterodine and its metabolites in general. In the context of the present application, the term “fesoterodine” therefore relates as a matter of principle to fesoterodine and/or its metabolites. “Metabolites” in this connection are understood to mean all substances formed during the metabolisation of fesoterodine, especially during metabolisation in the human body.

The metabolites are preferably fesoterodine 5-HM according to the following structure (2):

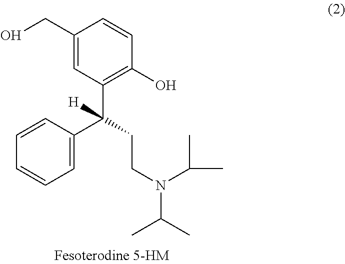

Since in the context of this application, the explanations regarding the active agent usually apply both to fesoterodine and to fesoterodine metabolites, the expression “fesoterodine (metabolite)” is also frequently used. As a matter of principle, the terms “fesoterodine” or “fesoterodine metabolite” in the context of this application comprise both the “free base” shown in structures (1) and (2) above and also pharmaceutically acceptable salts thereof. These may be one or more salts, which may also be present in a mixture. “Salt” is understood in this context to mean that the amine group of fesoterodine or the fesoterodine metabolite has been protonated, resulting in the formation of a positively charged nitrogen atom, which is associated with a corresponding counter-anion. The corresponding salts are also referred to in the context of this application as “fesoterodine (metabolite) salts”. In addition, in the context of this application, the terms fesoterodine, fesoterodine 5-HM and fesoterodine (metabolite) also encompass the enantiomers of the compounds shown in formulae (1) and (2) compounds.

The salts used are preferably acid addition salts. Examples of suitable salts are hydrochlorides, carbonates, hydrogen carbonates, acetates, lactates, butyrates, propionates, sulphates, methane sulphonates, citrates, fumarates, hydrogen fumarates, tartrates, maleinate, nitrates, sulphonates, oxalates and/or succinates.

In the case of fesoterodine or fesoterodine metabolite, it is particularly preferable that the pharmaceutically acceptable salt should be hydrogen fumarate. Hydrogen fumarate is a compound according to the formula HOOC—CH═CH—COO⁻, where the double bond has an E-configuration. In addition, in the case of fesoterodine or fesoterodine metabolite, it is particularly preferable that the pharmaceutically acceptable salt should be fumarate. Fumarate is a compound according to the formula —OOC—H═CH—COO⁻, where the double bond has an E-configuration.

It is likewise particularly preferable that the pharmaceutically acceptable salt should be tartrate, i.e. a salt of tartaric acid. Tartaric acid is also known in the art as 2,3-dihydroxy succinic acid. In the context of this invention, tartaric acid can be used as D-(−)-tartaric acid, L-(+)-tartaric acid, meso-tartaric acid or any mixture thereof, e.g. as the DL-racemate.

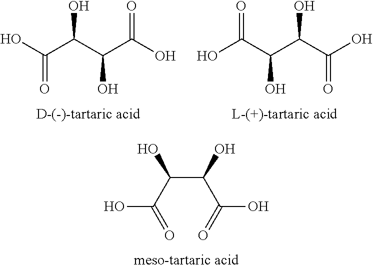

In a preferred embodiment, L-(+)-tartaric acid is used.

In the fesoterodine (metabolite) salt of the invention, tartaric acid may be present as a doubly (tartrate) or singly (hydrogen tartrate) negatively charged anion. The tartaric acid is preferably present as tartrate. It is accordingly possible for the molar ratio of fesoterodine (metabolite) to tartaric acid to be 1:1 to 2:1. In the fesoterodine (metabolite) salt of the invention, the molar ratio of fesoterodine (metabolite) to tartaric acid is preferably about 2:1.

In principle, the fesoterodine (metabolite) salt of the invention may be present, for example, in amorphous form, crystalline form or in the form of a solid solution. The fesoterodine (metabolite) salt of the invention is preferably present in crystalline form.

Hence, in the context of this invention, fesoterodine hydrogen fumarate, fesoterodine fumarate, fesoterodine tartrate, fesoterodine 5-HM-hydrogen fumarate (i.e. the compound according to formula (2) in the form of the hydrogen fumarate salt), fesoterodine 5-HM-fumarate (i.e. the compound according to formula (2) in the form of the fumarate salt), fesoterodine 5-HM-tartrate (i.e. the compound according to formula (2) in the form of the tartrate salt), fesoterodine 5-HM-hydrogen tartrate (i.e. the compound according to formula (2) in the form of the hydrogen tartrate salt) or mixtures thereof are preferably used as the active agent. In particular, fesoterodine fumarate is used. In particular, fesoterodine 5-HM-tartrate is used.

For the core, it is preferable to use fesoterodine and/or fesoterodine metabolite with a water content of 0.1 to 5% by weight, more preferably 0.3 to 3% by weight. The water content is determined by coulometric Karl Fischer titration, and preferably by means of the “Oven Sample Processor 774” as described in Metrohm, Application Bulletin 280/1d.

Fibers (b) are generally understood to mean substances which may usually be contained in foodstuffs but are not digestible in the gastrointestinal tract. It may be natural or synthetic fibers. Natural fibers are preferred. “Natural” here is understood to mean fibers based on naturally occurring components, wherein the components may be chemically modified.

One example of suitable synthetic fibers are ion exchange resins. An ion exchange resin is a polymer with which dissolved ions can be replaced by ions with the same type of charge. More preferably, a cation exchange resin is used. A cation exchange resin is a polymer containing functional groups with a cation that can be dissociated. Examples of these functional groups are sulphonic acid groups/sulphonate groups or carboxyl groups/carboxylate groups. Hence, as synthetic fibers (b), it is preferable to use a polymer that contains carboxyl groups/carboxylate groups and/or sulphonyl groups/sulphonate groups. If carboxylate or sulphonate groups are present, ammonium, alkali and alkaline earth ions, for example, may serve as counter-ions, with sodium and potassium, especially potassium, being preferred.

In a preferred embodiment, the synthetic fibers (b) are a copolymer obtainable by the copolymerisation of methacrylic acid and divinyl benzene. A copolymer of this kind is known under the designation polacrilin. In particular, in the context of this invention, polacrilin is used in the form of the potassium salt (polacrilin potassium, especially as monographed in accordance with the US Pharmacopoeia).

Polacrilin potassium can be illustrated by the following structural formula.

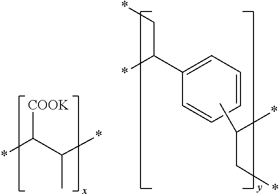

where x and y are natural numbers, such as 10¹ to 10²⁰, preferably 10⁶ to 10¹⁸. The ratio of x to y is usually 50:1 to 1:1, preferably 20:1 to 2:1, particularly preferably 10:1 to 3:1.

In a preferred embodiment, the fibers (b) consist of natural fibers. These are preferably vegetable fibers, i.e. substances that can be obtained from plants. More preferably, these are vegetable fibers with a gelling capacity (i.e. when these fibers are added to water, the viscosity increases, and preferably a gel forms.)

In a preferred embodiment, the fibers (b) have a gel strength (also known in English as “bloom strength”) of 5 to 500 g, more preferably 30 to 300 g, even more preferably 50 to 250 g, particularly preferably 60 to 200 g, especially 80 to 150 g.

The “gel strength” is a measure of the strength, or solidity, of a gel produced from a 6.67% by weight solution (consisting of fibers and water). The figures given above describe the mass needed to depress a defined surface of a gel by 4 mm. In the context of this application, the gel strength is determined in accordance with the official method of the “Gelatine Manufacturers Institute of America” (abbr. “GMIA”). On this subject, reference is made to the “GMIA Standard Methods for The Testing Of Edible Gelatine”, September 2006. For this purpose, a Brookfield Engineering “LFRA Texture Analyzer” is used, which has a punch with a diameter of 0.5″ (0.5 inches) and non-chamfered corners.

In a preferred embodiment, the fibers (b) do not contain any cellulose or cellulose derivatives such as cellulose esters or cellulose ethers. In particular, the fibers (b) are free of methyl cellulose, methyl ethyl cellulose, hydroxyethyl cellulose, hydroxypropyl methyl cellulose, hydroxypropyl cellulose or carboxymethyl cellulose or mixtures thereof. Similarly, the composition of the invention preferably likewise does not contain the above-mentioned cellulose derivatives or cellulose.

In addition, in a preferred embodiment, the component (b), or more preferably the pharmaceutical composition, does not contain any polyvinyl pyrrolidone, pregelatinised starch, polymethacrylate, polyvinyl acetate, dextran, starch or mixtures thereof.

In a particularly preferred embodiment, the fibers (b) are alginate, gelatine, agar, gum arabic, gum tragacanth, xanthan and carrageenan. In particular, kappa-carrageenan is used as fibers (b).

The individual types of fibers (b) will be explained in more detail below.

### Agar:

Agar (E 406) is usually found in the cell wall of red algae (Rhodophyceae), usually in the form of calcium and magnesium salts. It is usually prepared by means of hot-water extraction, purification and subsequent drying.

Agar preferably contains two fractions: agarose and agaropectin. The proportion of agarose is usually 40 to 75% by weight, preferably 55 to 66% by weight of the total weight. The proportion of agarose is substantially responsible for the gelling capacity. It is generally a neutral chain-like polysaccharide, in which D-galactose and 3,6-anhydro-L-galactose are linked together alternately in a β-1,4 and α-1,3-glycosidic linkage. Agaropectin usually has the same basic structure as agarose, but usually contains up to 10% sulphate groups, D-glucuronic acid and optionally pyruvic acid.

Preferably, agar with a weight-average molecular weight of 5,000 to 160,000 g/mol, more preferably 10,000 to 130,000 g/mol, is used. In the context of this invention, the weight-average molecular weight is determined by means of gel permeation chromatography.

Agar is particularly preferred fiber (b).

### Alginates:

Alginates (E 401) are salts of alginic acid (E 400). Alginic acid is preferably a linear polysaccharide, built up from D-mannuronic acid and L-guluronic acid, which are linked together β-1,4 glycosidically. Preferably, ammonium alginate (E 403), calcium alginate (E 404), potassium alginate (E 402) and/or sodium alginate (E 401) are used. It is usually obtained from seaweed (kelp)—mainly Macrocystis pyrifera and Laminaria species are used. Alginic acid is usually extracted with alkali and then the corresponding salts are precipitated in the acids.

Preferably, alginates are used with a weight-average molecular weight of 20,000 to 240,000 g/mol, more preferably 30,000 to 180,000 g/mol.

### Carrageenan:

Carrageenan (E 407) is the term usually used to describe the—preferably purified and dried—extracts of red seaweed (Rhodophyceae). The genera used to obtain carrageenan are preferably Chondrus crispus, Gigartina stellata and, to an increasing extent, Eucheuma cottonii and Eucheuma spinosa. In a dried form, these raw materials are also called carrageen (Irish moss).

For its production, the purified red algae are preferably extracted with hot water or alkalinically. The extract is either dried directly or mixed with alcohol to precipitate the carrageenan.

Preferred embodiments of carrageenan are lambda (λ) carrageenan, kappa (|) carrageenan and iota (┌) carrageenan.

Lambda carrageenan is a chain molecule built up of dimeric components, β-D-galactosido(1,4)-α-D-galactose. These dimers are linked together 1,3-glycosidically. The primary alcohol group of α-D-galactose is preferably esterified with sulphuric acid. The hydroxyl groups on the C-2 of both galactoses are esterified with sulphuric acid, preferably up to about 70%. Lambda carrageenan preferably has a sulphate content of between 25 and 45%, more preferably between 32 and 39%.

Lambda carrageenan preferably has the following structural unit:

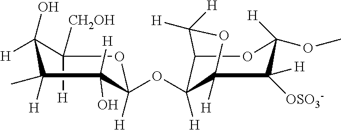

Kappa carrageenan is usually built up from the dimer carrabiose, in which β-D-galactose is 1,4-glycosidically linked to α-D-3,6-anhydrogalactose. These dimers are linked together into a chain molecule by 1,3-glycosidic linkages. |-carrageenan is partially sulphated; there is preferably a sulphate ester-group on C-4 of the galactose; kappa carrageenan preferably has a sulphate content of between 20 and 35%, more preferably between 25 and 30%.

Kappa carrageenan preferably has the following structural unit:

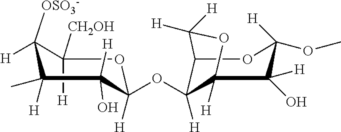

Kappa carrageenan is preferred as fibers (b) in the context of this invention.

The structure of iota carrageenan corresponds substantially to that of kappa carrageenan, where in addition, the hydroxyl group on the C-2 of anhydrogalactose can be esterified with sulphuric acid. The sulphate content is usually between 28 and 35%.

Iota carrageenan preferably has the following structural unit:

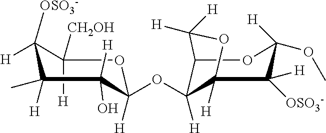

Carrageenans with a weight-average molecular weight of 80,000 to 850,000 g/mol, more preferably 120,000 to 750,000 g/mol, are preferably used.

Carrageenans may be present in the form of salts, e.g. in the form of potassium, sodium or calcium salts.

### Gelatine:

Gelatine is usually obtained by the selective hydrolysis of collagen, (a component of the connective tissue of animal skin and bones). The starting material that can be used is, for example, bones, pieces of hide and pigskin. The raw materials are usually precleaned and optionally have the fat removed. Bones are usually decalcified in addition, to leave ossein. After that, the collagen is usually swollen by treatment with an acid or alkali, and the gelatine is extracted in the heat in an acid environment.

Gelatine is usually a linear protein, preferably amphoteric in character. The weight-average molecular weight is usually 10,000 to 100,000, preferably 15,000 to 90,000 g/mol. Gelatine preferably contains the amino acids glycine (20 to 30%), proline (14 to 24%), hydroxyproline (10 to 18%), alanine (8 to 16%), aspartic acid (7 to 14%), arginine (6 to 11%), glutamic acid (4 to 8%), lysine (3 to 7%), leucine (3 to 7%) and serine (2 to 5%).

### Gum Arabic:

Gum arabic (E 414) can be obtained from exudate gum, i.e. from dried plant sap. Gum arabic is a acidic, branched polysaccharide, which can exist, for example, in the form of mixed potassium, magnesium and calcium salts. As monomeric building blocks, the free acid (arabic acid) usually contains D-galactose, L-arabinose, L-rhamnose, D-glucuronic acid.

Preferably, gum arabic with a weight-average molecular weight of 100,000 to 400,000 g/mol, more preferably 200,000 to 300,000 g/mol is used.

### Gum Tragacanth:

Gum tragacanth (E 413) can be obtained from the sap of Astragalus shrubs. Gum tragacanth is a branched polysaccharide, containing D-galacturonic acid, L-arabinose, D-galactose, L-fucose and D-xylose. Preferably, gum tragacanth with a weight-average molecular weight of 500,000 to 1,000,000 g/mol, more preferably 700,000 to 900,000 g/mol, used.

### Xanthan Gum:

Xanthan gum (E 415) is an extracellular polysaccharide of microbial origin. It can be obtained by fermentation using Xanthomonas campestris and subsequent alcohol precipitation of the culture filtrate. Xanthan gum contains D-glucose, D-mannose and D-glucuronic acid, preferably approximately in the ratio 2:2:1. Preferably, xanthan gum with a weight-average molecular weight of 500,000 to 3,000,000 g/mol, more preferably 800,000 to 2,000,000 g/mol, is used.

Xanthan can be used as, for example, sodium, calcium and/or potassium salt.

Apart from the above-mentioned fibers (b), it is also possible to use galactomannans. Galactomannans are the endosperm of seeds of different species of legumes. Endosperms are usually ground into flours.

The preferred galactomannans (which differ above all in the ratio of mannose/galactose), are locust bean gum (carobin, locust bean gum E 410), preferably mannose/galactose approx. 4:1, which is preferably obtainable from the seeds of Ceratonia siliqua;

guar gum (guaran E 412), preferably mannose/galactose approx. 2:1, which is preferably obtainable from the seeds of Cyamopsis tetragonolobus and C. psoralioides and tara gum (tara, E 417), preferably mannose/galactose approx. 3:1, which is preferably obtainable from seeds of Caesalpinia spinosa.

Tamarind can also be used as component (b). Tamarind is usually a hydrocolloid obtainable from the seeds of the tamarind (Tamarindus indica), containing 1,4-linked D-glucose units in the main chain and D-xylose, D-galactose and L-arabinose in the branches. The weight-average molecular weight is preferably 20,000 to 80,000, more preferably 30,000 to 70,000 g/mol.

In addition, karaya can also be used as component (b). Karaya (E 416) is an exudate gum obtainable from plant saps. It is preferably obtained from Sterculia species, specifically Sterculia urens or from Cochlospermum. Karaya contains acetylated polysaccharide, which comprises in particular D-galactose, L-rhamnose, D-galacturonic acid and D-glucuronic acid.

In a preferred embodiment, the term “fibers” does not comprise microcrystalline cellulose, calcium phosphate, especially calcium hydrogen phosphate dihydrate, sodium starch glycolate, magnesium stearate and/or colloidal silica. In addition, the term “fibers” preferably does not comprise polyvinyl pyrrolidone, pectin, polyacrylates, e.g. acrylate polymers known as Carbopol®, cellulose, cellulose derivatives, chitosan and polyoxyethylene. Furthermore, the fibers are preferably not selected from sorbitol, xylitol, polydextrose, isomalt, dextrose and/or hydroxypropyl methyl cellulose. In addition, the fibers are preferably not selected from polyvinyl pyrrolidone, cellulose ethers, such as hydroxyethyl cellulose and hydroxypropyl cellulose, cellulose esters, such as methyl cellulose, methyl ethyl cellulose, hydroxypropyl methyl cellulose, carboxymethyl cellulose, starch, pregelatinised starch, polymethacrylates, polyvinyl acetates, microcrystalline cellulose and/or dextrans.

Fesoterodine (metabolite) (a) and fibers (b) are usually employed as particulate solids. In this case, the average particle diameter (D50) is 1 to 500 μm, preferably 10 to 250 μm, more preferably 15 to 150 μm, particularly preferably 20 to 120 μm, especially 25 to 90 μm. It is preferable that fesoterodine (a) and fibers (b) should form a monomodal particle size distribution, especially with a view to achieving an advantageous content uniformity.

Unless anything else is specified, the expression “average particle diameter” relates in the context of this invention to the D50 value of the volume-average particle diameter determined by means of laser diffractometry. In particular, a Malvern Instruments Mastersizer 2000 was used to determine the diameter (wet measurement, 2,000 rpm, liquid paraffin as dispersant, ultrasound 60 sec., the evaluation being performed according to the Fraunhofer method). The average particle diameter, which is also referred to as the D50 value of the integral volume distribution, is defined in the context of this invention as the particle diameter at which 50% by weight of the particles have a smaller diameter than the diameter which corresponds to the D50 value. Similarly, 50% by weight of the particles then have a larger diameter than the D50 value.

In a preferred embodiment of the invention, the weight ratio of components (a):(b) is in the range from 1:50 to 1:2, more preferably 1:30 to 1:3, even more preferably 1:20 to 1:4, especially 1:15 to 1:5 and particularly preferably 1:12 to 1:8. In addition to (b), the pharmaceutical formulation of the invention may also comprise further pharmaceutical excipients. These are the excipients with which the person skilled in the art is familiar, such as those which are described in the European Pharmacopoeia. Examples of excipients used are disintegrants, tableting aids, anti-stick agents, additives to improve the powder flowability, glidants, wetting agents and/or lubricants. In a further preferred embodiment, the pharmaceutical formulation of the invention additionally contains acidifiers.

In a preferred embodiment, the pharmaceutical formulation of the invention contains

a) 0.1 to 20% by weight, more preferably 0.5 to 10% by weight fesoterodine and/or fesoterodine metabolites;
b) 0.5 to 80% by weight, more preferably 15 to 60% by weight fibers;
c) 0 to 15% by weight, more preferably 0.2 to 5% by weight disintegrant; and
d) 0 to 80% by weight, more preferably 25 to 75% by weight tableting aid, based on the total weight of the formulation.

In a further preferred embodiment, the pharmaceutical formulation of the invention also contains

(e) 0 to 35% by weight, preferably 5 to 25% by weight, acidifier.

The formulation of the invention may contain disintegrants (c). “Disintegrants” is the term generally used for substances which accelerate the disintegration of a dosage form, especially a tablet, after it is placed in water. Suitable disintegrants are, for example, organic disintegrants such as sodium carboxymethyl starch, croscarmellose and crospovidone. Alternatively, alkaline disintegrants are used. The term “alkaline disintegrants” means disintegrants which, when dissolved in water, produce a pH level of more than 7.0 e.g. NaHCO₃ or Na₂CO₃.

Sodium carboxymethyl starch is preferably used as the disintegrant.

The formulation of the invention may contain tableting aids (d). Tableting aids are understood to mean substances which have a filler effect and/or a binder effect. “Fillers” generally means substances which serve to form the body of the tablet in the case of tablets with small amounts of active agent. This means that fillers “dilute” the active agents in order to produce an adequate tableting mixture. The usual purpose of fillers, therefore, is to obtain a suitable tablet size.

Examples of preferred tableting aids are lactose, sucrose, microcrystalline cellulose (e.g. Avicel®), starch, pregelatinised starch (e.g. Starch 1500®), calcium phosphate, calcium carbonate, magnesium carbonate, magnesium oxide, calcium sulphate, hydrogenated vegetable oil, dextrin, cyclodextrin, and kaolin. Silicified microcrystalline cellulose can likewise be used. The silicified microcrystalline cellulose preferably used is commercially obtainable under the trade name Prosolv® and has a silica content of 1 to 3% by weight, preferably 2% by weight. Sucrose or pregelatinised starch is preferably used as the tableting aid.

One example of an additive to improve the powder flowability is disperse silicon dioxide, e.g. known under the trade name Aerosil®. Additives to improve the powder flowability are generally used in an amount of 0.1 to 3% by weight, based on the total weight of the formulation.

Lubricants can be used in addition. Lubricants are generally used in order to reduce sliding friction. In particular, the intention is to reduce the sliding friction found during tablet pressing between the punches moving up and down in the die and the die wall, on the one hand, and between the edge of the tablet and the die wall, on the other hand. Suitable lubricants are, for example, stearic acid, adipic acid, sodium stearyl fumarate (known by the trade name Pruv®) and/or magnesium stearate. Sodium stearyl fumarate is particularly preferred.

Lubricants are generally used in an amount of 0.1 to 3% by weight, based on the total weight of the formulation.

As acidifiers it is common to use substances which, when dissolved in water, lead to a pH of less than 7.0. Acidifiers are preferably compounds, especially organic compounds, which have at least one acid group. The compounds containing one or more acid group(s) preferably have a pKs value of 1.0 to 6.8, more preferably 1.8 to 6.6, even more preferably 2.8 to 6.4. The compounds may be present as the free acid or the salt. In the case of salts, alkaline or alkaline earth salts are preferred, especially sodium or potassium salts.

Examples of suitable acidifiers are adipic acid, malic acid, ascorbic acid, succinic acid, citric acid, fumaric acid, glutaric acid, maleic acid, malonic acid, tartaric acid and/or salts thereof. Examples of preferred salts are sodium citrates e.g. monosodium citrate or disodium citrate, sodium fumarate, potassium tartrate and/or sodium dihydrogen phosphate dihydrate. It is particularly preferable to use sodium citrate, especially sodium monocitrate, especially in the form of the dihydrate.

The pharmaceutical composition of the invention contains acidifiers usually in an amount from 0 to 35% by weight, more preferably 5 to 25% by weight, especially 7 to 17% by weight, based on the total weight the composition.

It lies in the nature of pharmaceutical excipients that they sometimes perform more than one function in a pharmaceutical formulation. In the context of this invention, in order to provide an unambiguous delimitation, the fiction will therefore preferably apply that a substance which is used as a particular excipient is not simultaneously also used as a further pharmaceutical excipient. Carrageenan, for example, if used as fibers (b), is then not also used as a disintegrant (c) (even though carrageenan also exhibits a certain disintegrating effect).

It is an advantage of the present invention that it is possible to dispense with moisture stabilisers. The pharmaceutical composition of the invention preferably does not contain any humectants, selected from glucose, glucose derivatives and sugar alcohols. In particular, the composition of the invention does not contain any humectants selected from isomalt, xylitol, sorbitol, polydextrose, dextrose or mixtures thereof.

The pharmaceutical composition of the invention can be processed into different oral dosage forms. It is preferably pressed into tablets. In a preferred embodiment, the composition of the invention is present in the form of a tablet, wherein the tablet is obtainable by direct compression. A suitable direct compression method will be explained in more detail below.

Alternatively, the composition of the invention (optionally after a granulation step) may be filled into capsules, sachets or stickpacks.

In a preferred embodiment, the pharmaceutical composition of the invention or the oral dosage forms of the invention are compositions or dosage forms with modified release. In the context of this invention, the expression “modified release” means delayed release, staggered release (repeat action release), prolonged release, sustained release or extended release. Prolonged release is preferable. In particular, the compositions or oral dosage forms of the invention have a release rate of less than 60% active agent after 2 hours. Furthermore, preferably less than 30% active agent after 1 hour. There is preferably an 85 to 100% release after 5 to 30 hours, especially after 10 to 25 hours. The release rate is preferably measured in accordance with USP, apparatus II (paddle), 500 ml test medium in phosphate puffer at pH 6.8, 37° C., 100 r.p.m.).

The pharmaceutical formulation of the invention is preferably used in the form of tablets. One subject matter of the invention is therefore a method of preparing a tablet containing the pharmaceutical formulation of the invention, comprising the steps of

(i) mixing
(a) fesoterodine and/or fesoterodine metabolites,
(b) fibers with pharmaceutical excipients, and optionally further pharmaceutical excipients,
(ii) compressing the mixture into tablets, optionally with the addition of further pharmaceutical excipients, and
(iii) optionally film-coating the tablets.

All the explanations provided above on preferred embodiments of the composition of the invention (e.g. on the type and quantity of components (a) and (b) and the further pharmaceutical excipients) also apply to the process of the invention. In addition to the process of the invention, tablets obtainable by means of the process of the invention are also a subject matter of the invention.

In step (i), components (a) and (b) and optionally further pharmaceutical excipients (as described above) are mixed. The mixing can be performed in conventional mixers. The mixing may, for example, be performed in compulsory mixers or free-fall mixers, e.g. using a Turbula® T 10B (Bachofen AG, Switzerland). The mixing time may, for example, be 1 minute to 10 minutes.

After mixing the resulting mixture can be screened. Screening is generally a procedure used to obtain an homogeneous powder mixture. By way of example, drum screens, vibration screens or conical screens (especially Quadro Comil®) can be used. It is preferable to use screens with a mesh width of 150 to 750 μm, especially 300 to 600 μm.

In step (ii), compression into tablets occurs. The compression can be performed with tableting machines known in the state of the art. The compression is preferably performed in the absence of solvents.

Examples of suitable tableting machines are eccentric presses or rotary presses. As an example, a Fette 102i® (Fette GmbH, Germany) can be used. In the case of rotary presses, a compressive force of 2 to 40 kN, preferably 2.5 to 35 kN, is usually applied. In the case of eccentric presses, a compressive force of 1 to 20 kN, preferably 2.5 to 10 kN, is usually applied. By way of example, the Korsch® EKO is used.

Process step (ii) is preferably performed in the absence of solvents, especially organic solvents, i.e. as dry compression.

In the optional step (iii) of the process of the invention, the tablets from step (ii) are film-coated. For this purpose, the methods of film-coating tablets which are standard in the state of the art may be employed.

For film-coating, macromolecular substances are preferably used, such as modified celluloses, polymethacrylates, polyvinyl pyrrolidone, polyvinyl acetate phthalate, zein and/or shellack.

The thickness of the coating is preferably 2 to 100 μm, more preferably 10 to 80 μm.

Furthermore, the tableting conditions in the method of the invention are preferably selected such that the resulting tablets have a ratio of tablet height to weight of 0.005 to 0.3 mm/mg, particularly preferably 0.05 to 0.2 mm/mg.

In addition, the resulting tablets preferably have a hardness of 50 to 200 N, particularly preferably 80 to 150 N. The hardness is determined in accordance with Ph. Eur. 6.0, section 2.9.8.

In addition, the resulting tablets preferably have a friability of less than 5%, particularly preferably less than 3%, especially less than 2%. The friability is determined in accordance with Ph. Eur. 6.0, section 2.9.7.

Finally, the tablets of the invention usually have a content uniformity of 90 to 110% of the average content, preferably 95 to 105%, especially 98 to 102%. The content uniformity is determined in accordance with Ph. Eur. 6.0, section 2.9.6.

The above details regarding hardness, friability, content uniformity and release profile preferably relate here to the non-film-coated tablet.

In an alternative embodiment, the tablets of the invention are prepared not by direct compression, but by means of dry granulation followed by pressing.

One aspect of the present invention therefore relates to a dry-granulation process comprising the steps of

(i-1) mixing fesoterodine (a) with fibers (b) and optionally further pharmaceutical excipients;
(i-2) compacting them into a slug;
(i-3) granulating the slug;
(ii) compressing the resulting granules into tablets, optionally with the addition of further pharmaceutical excipients; and
(iii) optionally film-coating the tablets.

In step (i-2) of the process of the invention, the mixture from step (i) is compacted into a slug. It is preferable here that it should be dry compacting, i.e. the compacting is preferably performed in the absence of solvents, especially in the absence of organic solvents. The compacting is preferably carried out in a roll granulator. The rolling force is preferably 5 to 70 kN/cm, preferably 10 to 60 kN/cm, more preferably 15 to 50 kN/cm. The gap width of the roll granulator is, for example, 0.8 to 5 mm, preferably 1 to 4 mm, more preferably 1.5 to 3 mm, especially 1.8 to 2.8 mm.

In step (i-3) of the process, the slug is granulated. Granulation can be performed with methods known in the state of the art. A Comil® U5 apparatus (Quadro Engineering, USA), for example, is used for granulating. In addition, the granulation conditions are preferably selected such that the resulting granules have a bulk density of 0.2 to 0.85 g/ml, more preferably 0.3 to 0.8 g/ml, especially 0.4 to 0.7 g/ml. The Hausner factor is usually in the range from 1.03 to 1.3, more preferably 1.04 to 1.20 and especially from 1.04 to 1.15. The “Hausner factor” in this context means the ratio of tapped density to bulk density. The tapped and bulk density are determined in accordance with Ph. Eur. 6.0, 2.9.15.

In a preferred embodiment, the granulation is performed in a screen mill. In this case, the mesh width of the screen insert is usually 0.1 to 5 mm, preferably 0.5 to 3 mm, more preferably 0.75 to 2 mm, especially 0.8 to 1.8 mm.

The compositions and oral dosage forms of the invention are preferably used for the treatment of an overactive bladder.

The subject matter of the invention is thus the use of vegetable fibers, selected from alginates, gelatine, agar, gum arabic, gum tragacanth, xanthan and carrageenan, for preparing a pharmaceutical formulation with modified release for the treatment of an overactive bladder. To put it another way, the subject matter of the invention is also a pharmaceutical formulation with modified release, containing vegetable fibers, selected from alginates, gelatine, agar, gum arabic, gum tragacanth, xanthan and carrageenan, for the treatment of an overactive bladder. All the explanations provided above on preferred embodiments of the composition of the invention (e.g. on the type and quantity of component (b) and the further pharmaceutical excipients) also apply to the use of the invention.

In a preferred embodiment of the use of the invention, a composition containing fibers (b) and acidifiers (e), and optionally (c) and (d) is used. Reference is made to the above explanations with regard to components (b) to (e) for detailed preferred embodiments.

In one preferred embodiment of the use of the invention, the pharmaceutical formulation contains one or more antimuscarinic agents. Examples of antimuscarinic agents are oxybutynin, solifenacin, fesoterodine, fesoterodine 5HM-metabolite, tolterodine and/or darifenacin.

The invention will now be illustrated with reference to the following examples.

### EXAMPLES

### Example 1

### Direct Compression

To prepare 200 tablets, 1.6 g fesoterodine fumarate, 30.0 g agar, 31.5 g dextrin and 0.6 g talcum were weighed in and mixed for 15 minutes (Turbula® T 10B). After that, 0.3 g sodium stearyl fumarate was added and mixed together with the other substances for a further 5 minutes (Turbula® T10B).

The tablets of 320 mg were compressed on a standard commercial eccentric press (Korsch® EKO) with a mould measuring 12.5×6.5 mm.

### Example 2

### Direct Compression

4 g fesoterodine fumarate were mixed for 10 minutes with 38.75 g agar and 36.38 g calcium phosphate (Turbula® T10B). The mixture was passed through a 500 μm screen, 0.5 g talcum and 0.38 g sodium stearyl fumarate were added, and the mixture was mixed for a further 5 minutes.

The finished mixture was used to produce 250 tablets of 320 mg on an eccentric press (Korsch® EKO).

### Example 3

### Dry Granulation

To prepare 160 tablets, 1.28 g fesoterodine fumarate and 25.6 g agar were granulated with 2.5 g water in a pharmaceutical mortar.

After drying for one hour at 40° C., the mixture was screened (Comil® U5) and then dried for one further hour.

23.4 g dextrin and 0.48 g talcum were added and the whole mixture was mixed for 10 minutes. Finally, 0.24 g sodium stearyl fumarate and 0.24 g sodium carboxymethyl starch were added, mixed for 3 minutes and then compressed on an eccentric press into tablets of 320 mg, the length being 12.5 mm and the width 6.5 mm.

### Example 4

### Direct Compression

| | | |
|---|---|---|
| Fesoterodine fumarate | 10.26 | mg |
| carrageenan, kappa | 100.00 | mg |
| talcum | 3.00 | mg |
| sucrose | 205.00 | mg |
| sodium stearyl fumarate | 1.50 | mg |
| sodium carboxymethyl starch | 1.50 | mg |

Fesoterodine fumarate, kappa carrageenan, talcum and sucrose were weighed in and mixed for 10 minutes (Turbula® T10B). After that, sodium stearyl fumarate and sodium carboxymethyl starch were added and mixed together with the other substances for 3 minutes. (Turbula® T10B). The tablets of 320 mg were compressed on an eccentric press (Korsch EKO).

### Example 5

### Direct Compression

| | | |
|---|---|---|
| Fesoterodine fumarate | 10.26 | mg |
| carrageenan, kappa | 100.00 | mg |
| pregelatinised starch | 165.00 | mg |
| talcum | 3.00 | mg |
| sodium citrate dihydrate | 40.00 | mg |
| sodium stearyl fumarate | 1.50 | mg |
| sodium carboxymethyl starch | 1.50 | mg |

Fesoterodine fumarate, kappa carrageenan, starch, talcum and sodium citrate dihydrate were weighed in and mixed for 10 minutes (Turbula® T10B). After that, sodium stearyl fumarate and sodium carboxymethyl starch were added and mixed together with the other substances for 3 minutes. (Turbula® T10B).

The tablets of 320 mg were compressed on an eccentric press (Korsch EKO).

### Example 6

### Direct Compression

| | | |
|---|---|---|
| Fesoterodine 5-HM-fumarate | 10.26 | mg |
| carrageenan, kappa | 100.00 | mg |
| pregelatinised starch | 165.00 | mg |
| talcum | 3.00 | mg |
| sodium citrate dihydrate | 40.00 | mg |
| sodium stearyl fumarate | 1.50 | mg |
| sodium carboxymethyl starch | 1.50 | mg |

Fesoterodine fumarate, kappa carrageenan, starch, talcum and sodium citrate dihydrate were weighed in and mixed for 10 minutes (Turbula® T10B). After that, sodium stearyl fumarate and sodium carboxymethyl starch were added and mixed together with the other substances for 3 minutes (Turbula® T10B).

The tablets of 320 mg were compressed on an eccentric press (Korsch EKO).

### Example 7

### Hydrolysis Behaviour

The hydrolysis behaviour of Examples 4 and 5 were investigated.

| | max. UI | Total | max. UI | Total | max. UI | Total |
|---|---|---|---|---|---|---|
| | 0 weeks | 0 weeks | 2 weeks | 2 weeks | 2 weeks | 2 weeks |
| Example 4 | 0.12 | 0.33 | 0.15 | 0.44 | 0.18 | 0.41 |
| Example 5 | 0.12 | 0.31 | 0.13 | 0.39 | 0.15 | 0.41 |

max. UI = maximum unknown impurity
Total = total of all impurities

It was thus shown that the composition of the invention leads to particularly small amounts of decomposition products. In particular, it was surprising that this could also be achieved by means of direct compression and even avoiding the use of stabilisers such as xylitol, since direct compression according to US 2008/0138421 has so far led to unsatisfactory stability results; rather, according to the US document, wet granulation was needed, cf. Table 8 of US 2008/0138421.

1. A pharmaceutical composition comprising
(a) fesoterodine and/or fesoterodine metabolites and
(b) fibers,
wherein the weight ratio of components (a):(b) is in the range from 1:50 to 1:2.

2. The pharmaceutical composition as claimed in claim 1, wherein component (a) is fesoterodine hydrogen fumarate, fesoterodine fumarate, fesoterodine tartrate, fesoterodine 5-HM-hydrogen fumarate, fesoterodine 5-HM-fumarate, fesoterodine 5-HM-tartrate and/or fesoterodine 5-HM-hydrogen tartrate.

3. The pharmaceutical composition as claimed in claim 1, wherein component (b) are vegetable fibers, preferably vegetable fibers with a gelling capacity.

4. The pharmaceutical composition as claimed in claim 1, wherein component (b) are free of cellulose or cellulose derivatives.

5. The pharmaceutical composition as claimed in claim 1, wherein the fibers have a gel strength of 50 to 300 g.

6. The pharmaceutical composition as claimed in claim 1, wherein component (b) are selected from alginates, gelatine, agar, gum arabic, gum tragacanth, xanthan and carrageenan.

7. The pharmaceutical composition as claimed in claim 1, wherein the composition additionally comprises an acidifier, preferably in an amount of 5 to 25% by weight, based on the total weight the composition.

8. The pharmaceutical composition as claimed in claim 1, wherein the pharmaceutical composition is free of humectants, selected from glucose, isomalt, xylitol, sorbitol, polydextrose and dextrose.

9. The pharmaceutical composition as claimed in claim 1, comprising
a) 0.1 to 20% by weight fesoterodine and/or fesoterodine metabolites;
b) 0.5 to 80% by weight fibers;
c) 0 to 15% by weight disintegrant; and
d) 0 to 80% by weight tableting aid.

10. The pharmaceutical composition as claimed in claim 1 in the form of a tablet, wherein the tablet is obtainable by direct compression.

11. The pharmaceutical composition as claimed in claim 1, wherein it is a composition with modified release.

12. A method of preparing tablets, comprising the steps of:
(i) mixing
(a) fesoterodine and/or fesoterodine metabolites, and
(b) fibers, with pharmaceutical excipients,
and optionally further pharmaceutical excipients to form a mixture,
(ii) compressing the mixture into tablets, optionally with the addition of further pharmaceutical excipients, and
(iii) optionally film-coating the tablets.

13. Tablets prepared by the method as claimed in claim 12.

14. A tablet with a friability of less than 3%, a content uniformity of 95 to 105% and a hardness of 50 to 180 N, comprising a pharmaceutical composition as claimed in claim 12.

15. A method for preparing a pharmaceutical formulation with modified release for the treatment of an overactive bladder, comprising the use if vegetable fibers, selected from alginates, gelatine, agar, gum arabic, gum tragacanth, xanthan and carrageenan.

16. The method as claimed in claim 15, wherein a composition comprising vegetable fibers and acidifiers is used.

17. The method as claimed in claim 15, wherein the pharmaceutical formulation comprises one or more antimuscarinic agents.
